(19) United States
(12) Patent Application Publication
(10) Pub. No.: US 2021/0353638 A1
(43) Pub. Date: Nov. 18, 2021

(54) TERPENE-ENRICHED CANNABINOID COMPOSITION AND METHOD OF TREATING CONDITIONS ASSOCIATED WITH AUTISM OR AUTISM SPECTRUM DISORDER

(71) Applicant: BUZZELET DEVELOPMENT AND TECHNOLOGIES LTD., OR-AKIVA (IL)

(72) Inventors: NOA RAZ, GIZO (IL); DANA BERNEMAN ZEITOUNI, KFAR YONA (IL); ISO HELLER, NETANYA (IL); AHARON EYAL, JERUSALEM (IL)

(21) Appl. No.: 17/386,569

(22) Filed: Jul. 28, 2021

Related U.S. Application Data
(63) Continuation-in-part of application No. PCT/IB2020/050635, filed on Jan. 28, 2020.
(60) Provisional application No. 62/844,279, filed on May 7, 2019.
(60) Provisional application No. 62/798,244, filed on Jan. 29, 2019.

(51) Int. Cl. A61K 31/55; A61K 31/4045; A61K 31/4458; A61K 31/05; A61K 31/352; A61K 45/06; A61K 36/68; A61K 36/23; A61K 36/81; A61K 36/41; A61K 31/138; A61K 31/27

(52) U.S. Cl. CPC A61K 31/55; A61K 31/4045; A61K 31/4458; A61K 31/05; A61K 31/352; A61K 31/27; A61K 36/68; A61K 36/23; A61K 36/81; A61K 36/41; A61K 31/138; A61K 45/06

(57) ABSTRACT

A terpene-enriched cannabinoid therapeutic product and methods for use thereof in treating conditions and/or symptoms associated with autism and/or autism spectrum disorder, the product including at least one cannabinoid, at least one primary terpene, and optionally at least three secondary terpenes, where the product has a therapeutic effect in treating conditions and/or symptoms associated with autism and/or autism spectrum disorder, and where said therapeutic effect is an enhanced therapeutic effect compared with that of a composition including the same cannabinoids amounts and one fifth the amount of said at least one primary terpene.

### FIELD

The invention relates generally to terpenes-enriched products, and specifically to terpenes-enriched products for therapeutic use.

### BACKGROUND

Terpenes play important roles in cannabinoids-comprising products, affecting the functionality and bioavailability of the cannabinoids and the aroma of the product. Processing cannabis plant material typically leads to terpenes loss so that most of cannabis products are of relatively low terpene content. That is particularly true for cannabis extracts and products thereof, such as cannabis tablets, cannabis gel capsules, cannabis patches, cannabis suppositories, etc. Most cannabis terpenes boiling points are in the range between about 150° C. and about 220° C. and they evaporate, at least partially, during cannabis buds drying, during solvent separation from extracts and during decarboxylation. Monoterpenes are lost at a rate greater than that of terpenes with a higher molecular weight and terpenes carrying no hydroxyl groups are lost at a rate greater than that of terpenes that do carry hydroxyl groups.

### SUMMARY

According to an aspect of some embodiments of the invention there is provided a method of treatment of a condition associated with autism and/or an autism spectrum disorder selected from the group consisting of restlessness, rage attacks, agitation, treatment-resistant self-injurious behavior, stress, aggression, anxiety, irritability, behavioral outbreaks, communication problems, disruptive behavior, psychosis, hyperactivity, lethargy, seizure activity, hypersensitivity, stereotypy, inappropriate speech, sleep problems, insomnia, cognitive impairment, motor and/or vocal tics, digestive problems, lack of appetite, constipation, depression, incontinence, difficulty in concentration, attention disorder, poor learning capabilities, poor daily functioning, Asperger's syndrome, a pervasive developmental disorder in a not otherwise specified (PDD-NOS) subject, Rett syndrome, an intellectual disability, a condition characterized by language impairment, a condition characterized by social and communication disabilities, a condition characterized by unusual behaviors and interests, nervousness, nausea and vomiting, fatigue, deficits in balance, and combinations thereof in a subject in need thereof, the method comprising administering to the subject a therapeutically effective amount of a composition comprising

(i) at least one cannabinoid; and

(ii) at least one primary terpene selected from the group consisting of limonene, eucalyptol, linalool, terpineol, caryophyllene, pinene, myrcene, humulene, citronellol, terpinen-4-ol, ocimene, geraniol, menthol, borneol, bisabolol, fenchol, guiaol and combinations thereof,

wherein said at least one primary terpene forms at least 20% of the total amount of terpenes in said composition,

wherein said at least one primary terpene optionally comprises a dedicated terpene which forms at least 40% by weight of the total amount of said primary terpene in the composition, and

wherein a total terpenes to cannabinoids weight/weight ratio in said composition is greater than 0.05:1.

According to some embodiments, said composition comprises a dedicated terpene selected from the group consisting of humulene, caryophyllene, bisabolol, guaiol, pinene, linalool, terpineol, limonene, eucalyptol and combinations thereof, wherein said condition occurs during the daytime, wherein said condition is selected from the group consisting of including behavioral outbreaks, aggression, rage attacks, self-injurious behavior, irritability, restlessness, disruptive behavior, psychosis, hyperactivity, hypersensitivity, stereotypy, inappropriate speech, motor and/or vocal tics, communication problems, attention disorder, poor learning capabilities and poor daily functioning.

According to some embodiments, said composition comprises a dedicated terpene selected from the group consisting of caryophyllene, bisabolol, pinene, linalool, terpineol and combinations thereof, wherein said condition comprises sleep problems.

According to some embodiments, said condition is selected from the group consisting of Asperger's syndrome, a pervasive developmental disorder in a not otherwise specified (PDD-NOS) subject, Rett syndrome, an intellectual disability, a condition characterized by language impairment, a condition characterized by social and communication disabilities, a condition characterized by unusual behaviors and interests, and combinations thereof.

According to some embodiments, said composition further comprises at least one selected from the group consisting of bergamottin, apigenin, amentoflavone, quercetin and piperine and combinations thereof.

According to some embodiments, said subject is a child.

According to some embodiments, said subject is a female.

According to some embodiments, said composition further comprises a selected amount of a herbal preparation produced by at least one of distilling and extracting, wherein said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, lycium barbarum, Matricaria, Melissa officinalis, Mentha piperita, Mitaki, Mumio, Olea europea, origanum, Paeonia lactiflora, panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to some embodiments, said at least one primary terpene is selected from the group consisting of limonene, eucalyptol, linalool, terpineol, myrcene, caryophyllene, citronellol, pinene, terpinene, nerolidol, eucalyptol, sabinene, pinene, limonene, borneol, fenchol, bisabolol, humulene, phytol, citral and combinations thereof, wherein said composition further comprises an herbal preparation selected from the group consisting of extracts of Mellisa, Bacopa, Valeriane, Avena Sativa, passiflora, Humulus Lopus and combinations thereof, and wherein said condition comprises insomnia and/or sleep problems.

According to some embodiments, said composition further comprises a compound selected from the group consisting of Mirtazapine, melatonin, Donepezil and combinations thereof.

According to some embodiments, said at least one cannabinoid comprises CBD and optionally THC at a CBD to THC weight/weight ratio greater than 1:1, wherein said primary terpene is selected from the group consisting of limonene, eucalyptol, linalool, terpineol, pinene, pulegone, cymene, ocimene, caryophyllene, humulene, carene and combinations thereof,

wherein said compositions further comprises an herbal preparation selected from the group consisting of extracts of Bacopa, Centella, Witania, Rhodiola and combinations thereof, and

wherein said condition is selected from the group consisting of difficulty in concentration, cognitive impairment and attention disorder and combinations thereof.

According to some embodiments, said composition further comprises a compound selected from the group consisting of Methylphenidate, Venlafaxine Memantine, rivastigmine and combination thereof.

According to some embodiments, said at least one cannabinoid comprises CBD and optionally THC at a CBD to THC weight/weight ratio greater than 1:1, wherein said primary terpene is selected from the group consisting of limonene, eucalyptol, linalool, terpineol, citronellol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene and combinations thereof,

wherein said composition further comprises an herbal preparation selected from the group consisting of extracts of Hypericum Perfortum, Avena Sativa, Passiflora, Humulus Lopus and combinations thereof, and said administering is performed during nightime; or

wherein said composition comprises an herbal preparation selected from the group consisting of extracts of Hypericum Perfortum, Centella, Bacopa, mellisa, Verbena officinalis and combinations thereof, and wherein said administering is performed during daytime, and

wherein said condition is selected from the group consisting of anxiety, irritability, nervousness, restlessness, stress, aggression, hyperactivity, disruptive behavior, psychosis, behavioral outbreaks, hypersensitivity, rage attack, agitation, treatment-resistant self-injurious behavior, communication problems, depression and combinations thereof.

According to some embodiments, said at least one cannabinoid comprises THC and optionally CBD at a THC to CBD weight/weight ratio greater than 1:1,

wherein said at least one primary terpene is selected from the group consisting of limonene, eucalyptol, linalool, terpineol, citronellol, geraniol, menthol, humulene, bisabolol, borneol, fenchol, pinene, caryophyllene, myrcene, cymene, terpinene and combinations thereof,

wherein said composition further comprises an herbal preparation selected from the group consisting of extracts of Atractylodes macrocephala, Angelica arc, Mentha piperita, Matricaria, Zingiber officinalis and combinations thereof, and

wherein said condition is selected from the group consisting of lack of appetite, nausea and vomiting and combinations thereof.

According to some embodiments, said at least one primary terpene is selected from the group consisting of limonene, eucalyptol, linalool, terpineol, cymene, ocimene, terpinene, caryophyllene, citronellol, sabinene, pinene, myrcene, germacrene, geraniol, nerolidol, menthol, humulene, fenchol, borneol and combinations thereof,

wherein said composition further comprises an herbal preparation selected from the group consisting of extracts of Aloe spp, Angelica arc, Rumex, Silybum marianum, Rehmania glutinosa, Taraxacum, Humulus lupus, Verbena officinalis and combinations thereof, and

wherein said condition is selected from the group consisting of digestive problems, incontinence and constipation and combinations thereof.

According to some embodiments, said primary terpene is selected from the group consisting of limonene, eucalyptol, linalool, terpineol, myrcene, pinene, nerolidol, caryophyllene, geraniol, menthol, borneol, bisabolol, fenchol and combinations thereof;

wherein said composition further comprises an herbal preparation selected from the group consisting of extracts of Cypripedium pubescens, whitania, Hypericum, Rhodiola, Vallerian, passiflora, Bacopa, Centella, Rhodiola and combinations thereof,

wherein said administering is performed during daytime, and

wherein said condition is selected from the group consisting of stereotypy, inappropriate speech, motor tics and/or vocal tics and combinations thereof.

According to some embodiments, said at least one cannabinoid comprises CBD and optionally THC at a CBD to THC weight/weight ratio greater than 1:1, wherein said primary terpene is selected from the group consisting of limonene, eucalyptol, linalool, terpineol ocimene, myrcene, limonene, caryophyllene and combinations thereof,

wherein said composition further comprises an herbal preparation selected from the group consisting of extracts of Bacopa, centella, Curcuma, Whitania, Zingiber officinalis, Peonia and combinations thereof, and

wherein said condition comprise seizure activity.

According to some embodiments, said at least one cannabinoid comprises CBD and optionally THC at a CBD to THC weight/weight ratio of greater than 1:1, wherein said primary terpene is selected from the group consisting of limonene, eucalyptol, linalool, terpineol pinene, terpinene, myrcene, caryophyllene, borneol and combinations thereof,

wherein said composition further comprises an herbal preparation selected from the group consisting of extracts of centella, Rhodiola, Whitania, Astragalus membranous, Bacopa, Ganoderma and combinations thereof,

wherein said condition is selected from the group consisting of lethargy, fatigue and deficits in balance and combinations thereof.

According to some embodiments, said composition comprises a compound selected from the group consisting of Mirtazapine, melatonin, Donepezil, Methylphenidate, Venlafaxine Memantine, rivastigmine, Risperidone, Aripiprazole, Clozapine, Haloperidol, Sertraline, Oxytocin, Secretin, Fluoxetine, Citalopram, Bumetanide, Acamprosate, Atomoxetine, baclofen, DMXB-A (a derivative of the nemertine toxin anabasein), EPI-743 (vincerinone), RG7314 (Roche), Divalproex, Dextromethorphan hydrobromide, and/or quinidine sulfate and combinations thereof.

### DETAILED DESCRIPTION

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

Unless indicated otherwise, the term “cannabinoid” as used herein refers to a compound that affects the endocannabinoid system. Cannabinoids are agonists or antagonists to receptors in the endocannabinoid system.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, the term “cellulose” refers to cellulose, hemicellulose and their combinations. As used herein, the term “glycol” refers to any glycol, including ethylene glycol, polyethylene glycol, propylene glycol and polypropylene glycol. As used herein, the term “chlorophyll” refers to chlorophyll and degradation products thereof.

As used herein, “terpene” refers to both terpenes and terpenoids.

As used herein and unless indicated otherwise, the term “terpenes/cannabinoids (or terpenes to cannabinoids) weight/weight ratio” means the weight ratio between the combined amount of terpenes and the combined amount of cannabinoids.

The particulars shown herein are by way of example and for purposes of illustrative discussion of the various embodiments of the invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of an embodiment of the invention, there is provided a terpene-enriched cannabinoid therapeutic product comprising
(i) at least one cannabinoid,
(ii) at least one primary terpene; and
(iii) optionally at least three secondary terpenes,
   wherein said product has an enhanced therapeutic effect in treating conditions and/or symptoms associated with autism and/or autism spectrum disorder, compared with that of a product comprising the same amount of cannabinoids and one fifth the amount of said primary terpene.

According to an embodiment, the product is a composition.

According to an embodiment, the composition comprises at least one cannabinoid, at least two cannabinoids, at least three, at least four or at least five. According to an embodiment, the content of each cannabinoid in the composition is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively) and cannabinol in acid or decarboxylated form (CBNa or CBN, respectively), Cannabicyclol in acid or decarboxylated form (CBLa or CBL, respectively). As used herein, the term “THC” refers to THCa (tetrahydrocannabiniolic acid) and/or to THC (tetrahydrocannabiniol) unless indicated otherwise. As used herein, the term “CBD” refers to CBDa (tcannabidiolic acid) and/or to CBD (cannabidiol) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise.

According to an embodiment, at least one of the cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the composition comprises THC and/or THCa. According to an embodiment, the composition comprises CBD and/or CBDa. According to an embodiment, the composition comprises THC and/or THCa at a content of less than 1%, less than 0.8%, less than 0.6%, less than 0.4% or less than 0.2%. According to an embodiment, the composition comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the composition comprises CBG and/or CBGa. According to an embodiment, the composition comprises CBN and/or CBNa. According to an embodiment, the composition comprises CBC and/or CBCa. According to an embodiment, the composition comprises CBL and/or CBLa. According to an embodiment, the composition comprises THCV and/or THCVa. According to an embodiment, the composition comprises CBDV and/or CBDVa.

According to an embodiment, the composition comprises a carrier. According to an embodiment, the composition comprises at least 2% by weight carrier, at least 3%, at least 5%, at least 10%, at least 15%, at least 20%, at least 25%, at least 30%, at least 35% or at least 40%, at least 50%, at least 60%, at least 70%, or at least 80% by weight carrier. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term “cellulose” refers to cellulose, hemicellulose and their combinations.

According to an embodiment, the composition comprises less than 5% by weight glycol, less than 4%, less than 3%, less than 2%, or less than 1%, by weight glycol. As used herein, the term “glycol” refers to any glycol, including ethylene glycol, polyethylene glycol, propylene glycol and polypropylene glycol.

According to an embodiment, the composition comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5%.

According to an embodiment, the composition comprises at least one primary terpene and optionally at least two secondary terpenes, at least three, at least four or at least five. According to an embodiment, the primary terpene comprises up to 8 terpenes, up to 7, up to 6, up to 5, up to 4, or up to 3 terpenes. The term “terpene”, as used herein, refers to both terpenes and terpenoids. As used here, the term “primary terpene” refers to a terpene that forms at least 20% by weight of the total amount of terpenes in the composition, at least 30%, at least 40%, at least 50%, at least 60%, at least 70% or at least 80%. According to an embodiment, the composition comprises multiple (e.g. two or three) terpenes, each one of which forms at least 20% by weight of the total amount of terpenes in the composition and each one of these terpenes is considered a primary terpene. As used here, the term “secondary terpene” refers to a terpene that forms at least 10 parts per million (ppm) of the composition. According to an embodiment, the content of the at least one primary terpene in the composition is at least 2 times greater than that of any secondary terpene, at least 4, at least 6, at least 8, at least 10, at least 15, at least 20, at least 25, or at least 30 times greater. According to an embodiment, the secondary terpene refers to a terpene that forms less than 1/10 of said primary terpene, less than ⅛, less than ⅕, or less than ½ of said primary terpene. According to an embodiment, the content of said primary terpene in said composition is at least 10 times greater than that of any secondary terpene. According to an embodiment, the total content of the all secondary terpenes is less than 20% of the total amount of terpenes in said composition.

As used herein, “terpenes/cannabinoids (or terpenes to cannabinoids) weight/weight ratio” means the weight ratio between the combined amount of terpenes and the combined amount of cannabinoids. According to an embodiment, the composition comprises at least 5% by weight cellulose and the terpenes to cannabinoids weight/weight ratio in the composition is in the range between about 0.1 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2 or less than 0.15. According to an embodiment, the composition comprising more than 5% cellulose is selected from the group consisting of cannabis plant material, e.g. cannabis buds or cannabis trim, ground forms thereof, plant material preparation for vaporizers and cannabis cigarette. According to an embodiment, the composition comprises a dried cannabis plant material.

According to another embodiment, the non-cannabinoid, non-terpene carrier comprises less than 5% by weight cellulose and terpenes to cannabinoids weight/weight ratio in the composition is in the range between about 0.05 and about 1.0. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2, less than 0.15 or less than 0.1. According to an embodiment, the composition comprising less than 5% cellulose is selected from the group consisting of cannabis trichomes, cannabis extracts and products thereof, such as tablets, gel capsules, medical patches, vaporizer liquids, mouthwashes, varnishes and suppositories.

According to an embodiment, the at least one primary terpene, the secondary terpenes or both are selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gurjunene, farnesene, friedelin, carvacrol, eugenol, geranyl acetate, camphor, menthol, iso-menthone, neral, gerial, viridiflorol, germacrene, thymol, Menth-2-en-1-ol, farensol, carotol, myrtenol isomers thereof and combinations thereof. According to an embodiment, the at least one primary terpene the secondary terpenes or both, are selected from the group consisting of pinene, limonene, linalool, caryophyllene, myrcene, terpinene, terpineol and combinations thereof. According to an embodiment, at least one of the terpenes is a-cyclic. According to an embodiment, at least one of the terpenes is cyclic. According to an embodiment, at least one of the terpenes is not found in cannabis buds or is present there at less than 0.2%, less than 0.1%, less than 0.05% or less than 0.02%. Such terpene is referred to as “non-cannabis terpene”. According to an embodiment, the terpene-enriched cannabinoid composition comprises the non-cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, the at least one primary terpene is selected from the group consisting of pinene, limonene, linalool, caryophyllene, myrcene, humulene, borneol, eucalyptol, terpineol, nerolidol, phytol, geraniol, bisabolol, camphene, beta-amyrin, thuj one, citronellol, pulegone, cycloartenol, cymene, sabinene, carene, terpinene, isopulegol, guaiol, phellandrene, eudesmol, ocimene, cardinene, elemene, gurjunene, farnesene, friedelin, carvacrol, eugenol, fenchol, geranyl acetate, camphor, menthol, iso-menthone, neral, gerial, viridiflorol, germacrene, thymol, Menth-2-en-1-ol, farensol, carotol, myrtenol and combinations thereof. According to an embodiment, the at least one primary terpene is selected from the group consisting of pinene, limonene, linalool, caryophyllene, myrcene and humulene and combinations thereof. According to an embodiment, the at least one primary terpene is a non-cannabis terpene.

According to an embodiment, the terpenes comprise at least one monoterpene selected from the group consisting of limonene, myrcene, pinene, linalool, geraniol, terpinolene camphene and isomers thereof and combinations thereof. According to an embodiment, the terpenes comprise at least one sesquiterpene selected from the group consisting of nerolidol, caryophyllene, farnesene, zingiberene, vetivazulene, guaiazulene, longifolene, copaene, patchoulol humulene and isomers thereof and combinations thereof. According to an embodiment, the terpenes comprise at least one diterpene selected from the group consisting of phytol, retinal, retinol, phytane, cembrene, sclarene, labdane, abietane, texadiene, stemarene, stemoden and isomers thereof and combinations therefo. According to an embodiment, the terpenes comprise at least one hydroxy-terpene selected from the group consisting of nerolidol, geraniol, linalool, phytol and isomers thereof and combinations thereof. As used herein “hydroxy-terpene” refers to a terpene carrying a hydroxyl function.

According to an embodiment, at least one of the terpenes is a monoterpene, at least one of the terpenes is a sesquiterpene and the monoterpenes to sesquiterpenes weight/weight ratio (i.e. the weight ratio between the total amount of monoterpenes and the total amount of the sesquiterpenes) is greater than 1.5 greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, at least one of the terpenes is a monoterpene, at least one of the terpenes is a diterpene and the monoterpenes to diterpenes weight/weight ratio (i.e. the weight ratio between the total amount of monoterpenes and the total amount of the diterpenes) is greater than 5, greater than 6, greater than 7, greater than 8, greater than 9, greater than 10, greater than 12, greater than 14, greater than 16, greater than 18, greater than 20, greater than 25, or greater than 30.

According to an embodiment, at least one of the terpenes carries no hydroxyl group, at least one of the terpenes carries hydroxyl group and the non-hydroxy-terpenes to hydroxyl-terpenes weight/weight ratio (i.e. the weight ratio between the total amount of non-hydroxy-terpenes and the total amount of the hydroxy-terpenes) is greater than 1.5, greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, terpenes form at least 20% by weight of the composition, at least 30%, at least 40%, at least 50%, at least 60% or at least 70%.

According to an embodiment, the composition is liquid at 30° C. According to an embodiment, the composition is a suspension at 30° C. According to an embodiment, the composition is essentially clear of haze or suspended solids at 30° C.

According to an embodiment, the composition comprises cannabis plant material. According to an embodiment, the composition comprises cannabis bud. According to an embodiment, the cannabis bud forms at least 20% of the composition, at least 30%, at least 40%, at least 50%, at least 60%, at least 70% or at least 80%.

According to an embodiment the composition comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the composition comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the composition comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and CBDc and/or CBDa in a total concentration of at least 10% by weight.

According to a related embodiment, the composition comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, the composition comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the composition comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the composition comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight. According to an embodiment, the composition comprises water and water content is more than 50%, more than 60%, more than 70% more than 80%, more than 90%, or more than 95%.

According to an embodiment, the composition comprises chlorophyll. According to an embodiment, the composition comprises at least 0.5% chlorophyll, at least 1, % at least 5%, at least 10%, at least 15%, at least 20% chlorophyll. As used herein, the term chlorophyll refers to chlorophyll and degradation products thereof. According to an embodiment, the composition comprises at least one flavonoid. According to an embodiment, the composition comprises at least two, at least three, at least four, or at least five flavonoids. —According to an embodiment, the composition comprises at least one of bergamottin, apigenin, amentoflavone, quercetin and piperine. According to an embodiment, the composition comprises at least two, at least three, or at least four of bergamottin, apigenin, amentoflavone, quercetin and piperine. According to an embodiment, the composition comprises bergamottin. According to an embodiment, the composition comprises apigenin. According to an embodiment, the composition comprises amentoflavone. According to an embodiment, the composition comprises quercetin. According to an embodiment, the composition comprises piperine.

According to an embodiment, the composition comprises a dried cannabis plant material. According to an embodiment, the composition comprises ground cannabis plant material, is a vaporizer cannabis filling material or is a cannabis cigarette.

According to an embodiment, the composition is liquid. According to an embodiment, the composition is selected from the group consisting of vaporizer cannabis filling liquid, cannabis tablets, cannabis suppositories, cannabis gel capsules, cannabis candies, cannabis drinks, cannabis mouthwashes, cannabis varnishes and cannabis baked products.

According to an embodiment, the composition further comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the composition comprises a surfactant selected from the group consisting of phospholipids, glycerides, glycolipids and combinations thereof. According to an embodiment, the composition further comprises a food-approved texturizer. According to an embodiment, the composition further comprises at least 10 ppm ethanol. According to an embodiment, the composition further comprises at least one of vitamin C, vitamin E, polyunsaturated fatty acids, beeswax and coconut oil. According to an embodiment, the composition further comprises a sweetener.

According to an embodiment, the composition is selected from the group consisting of cannabis tablets, cannabis gel capsules, cannabis medical patches, cannabis cigarettes and cannabis vaporizer liquids, cannabis suppositories, cannabis candies, cannabis drinks, cannabis mouthwashes, cannabis varnishes and cannabis baked products.

According to an embodiment, the composition provides a therapeutic effect which is an enhanced therapeutic effect compared with that of a composition comprising the same cannabinoids amounts and a smaller amount of the at least one primary terpene, e.g. one half, one third, one quarter or one fifth of that amount. According to various embodiment, the increased therapeutic effect has various forms, e.g. a shortened onset time of the therapeutic effect, an increased magnitude of the therapeutic effect, an extended duration of the therapeutic effect, a reduced requirement for the composition, a reduction in at least one secondary adverse symptom, a reduced frequency of at least one of said conditions and/or symptoms, a reduced severity of at least one of said conditions and/or symptoms, a reduced consumption of other drugs and combinations thereof. According to an embodiment, the increased therapeutic effect comprises a shorter onset time, or differently put, an earlier effect, which is important particularly in cases of sublingual and topical delivery and in cases where a rapid effect is desired, as in treating trauma patients. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main condition, of another condition and/or conditions related to administration of the composition or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, the composition comprises less than 5% by weight glycol.

According to an embodiment, the composition comprises less than 20% by weight water.

According to an embodiment, the onset time of the therapeutic effect, as measured by methods known in the art, is a time at least 20% shorter than the onset time obtained by administering a composition comprising the same cannabinoids amounts and one fifth the amount of the at least one primary terpene, in some embodiments at least 30%, at least 40%, at least 50% or at least 60% shorter. According to an embodiment, wherein the composition comprises at least two secondary terpenes, the onset time of the therapeutic effect is at least 20% shorter than that of a composition comprising the same cannabinoids amounts, same secondary terpene amounts and one fifth the amount of the at least one primary terpene, at least 30%, at least 40%, at least 50% or at least 60% shorter. Shorter onset time, or differently put an earlier effect, is important particularly in cases of sublingual and topical delivery and in cases where a rapid effect is desired, as in treating pain.

According to an embodiment, the composition provides an onset time of the therapeutic effect which is at least 20% longer than that obtained by administering a composition comprising the same cannabinoids amounts and one fifth the amount of the at least one primary terpene, in some embodiments at least 30%, at least 40%, at least 50% or at least 60% longer. According to an embodiment, wherein the composition comprises at least two secondary terpenes, the onset time of the therapeutic effect is at least 20% longer than that obtained by administering a composition comprising the same cannabinoids amounts, same secondary terpene amounts and one fifth the amount of the at least one primary terpene, in some embodiments at least 30%, at least 40%, at least 50% or at least 60% longer. According to an embodiment, compositions providing delayed onset time are used in combination with shorter onset time to reach a sustained release effect.

According to an embodiment, the magnitude of the therapeutic effect, as measured by methods known in the art, is at least 20% greater than that obtained by administering a composition comprising the same cannabinoids amounts and one fifth the amount of the at least one primary terpene, in some embodiments at least 30%, at least 40%, at least 50% or at least 60% greater. According to an embodiment, wherein the composition comprises at least two secondary terpenes, and the magnitude of the therapeutic effect is at least 20% greater than that obtained by administering a composition comprising the same cannabinoids amounts, same secondary terpene amounts and one fifth the amount of the at least one primary terpene, in some embodiments at least 30%, at least 40%, at least 50% or at least 60% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, the duration of the therapeutic effect, as measured by methods known in the art, is at least 20% longer than that obtained by administering a composition comprising the same cannabinoids amounts and one fifth the amount of the at least one primary terpene, in some embodiments at least 30%, at least 40%, at least 50% at least 60%. at least 70%, at least 80%, at least 90% or at least 100% longer. According to an embodiment, wherein the composition comprises at least two secondary terpenes, the duration of the therapeutic effect is at least 20% longer than that obtained by administering a composition comprising the same cannabinoids amounts, same secondary terpene amounts and one fifth the amount of the at least one primary terpene, in some embodiments at least 30%, at least 40%, at least 50%, at least 60%. at least 70%, at least 80%, at least 90% or at least 100% longer.

According to an embodiment, the frequency of the conditions and/or symptoms, as measured by methods known in the art, is at least 20% smaller compared with that obtained by administration of a composition comprising the same cannabinoids amounts and one fifth the amount of the at least one primary terpene, at least 30%, at least 40%, at least 50% at least 60%. at least 70%, at least 80%, or at least 90% smaller. According to an embodiment, wherein the composition comprises at least two secondary terpenes, the frequency of the conditions and/or symptoms, as measured by methods known in the art, is at least 20% smaller than that obtained by administering a composition comprising the same cannabinoids amounts, same secondary terpene amounts and one fifth the amount of the at least one primary terpene, in some embodiments at least 30%, at least 40%, at least 50%, at least 60%. at least 70%, at least 80%, at least 90% or at least 100% longer.

According to an embodiment, the severity of the conditions and/or symptoms, as measured by methods known in the art, is at least 20% smaller than that obtained by administration of a composition comprising the same cannabinoids amounts and one fifth the amount of the at least one primary terpene, at least 30%, at least 40%, at least 50% at least 60%. at least 70%, at least 80%, or at least 90% smaller. According to an embodiment, wherein the composition comprises at least two secondary terpenes, the severity of the conditions and/or symptoms, as measured by methods known in the art, is at least 20% smaller than that of a composition comprising the same cannabinoids amounts, same secondary terpene amounts and one fifth the amount of the at least one primary terpene, in some embodiments at least 30%, at least 40%, at least 50%, at least 60%. at least 70%, at least 80%, at least 90% or at least 100% longer.

According to an embodiment, the composition is for administration in a vaporizer. According to an embodiment, the composition is for sublingual administration. According to an embodiment, the composition is for topical administration. According to an embodiment, the composition is for administration as a suppository (vaginal or rectal). According to an embodiment, the composition is for administration as a mouthwash. According to an embodiment, the composition is for administration as a varnish.

According to an embodiment, provided is a terpene-enriched cannabinoid therapeutic product comprising (i) at least one cannabinoid in a specific amount, (ii) a primary terpene in a specific amount, and (iii) optionally at least three secondary terpenes, wherein said primary terpene forms at least 20% of the total amount of terpenes in said product and wherein said product has an enhanced therapeutic effect in treating conditions and/or symptoms associated with autism and/or autism spectrum disorder, compared with that of a product comprising the same amount of cannabinoids and one half the amount of said primary terpene.

According to an embodiment, said primary terpene forms at least 20% of the total amount of terpenes in said product, at least 25%, at least 30%, at least 35%, at least 40%, at least 45%, at least 50%, at least 55% or at least 60% of the total amount of terpenes in said product.

According to an embodiment, said product has an enhanced therapeutic effect in treating at least one of restlessness, rage attack, agitation, treatment-resistant self-injurious behavior, stress, aggression, anxiety, irritability, behavioral outbreaks, communication problems, disruptive behavior, psychosis, hyperactivity, lethargy, seizure activity, hyper sensitivity, stereotypy, inappropriate speech, sleep problem, cognitive impairment, motor and/or vocal tics, digestive problems, lack of appetite, constipation, depression, incontinence, difficulty in concentration, attention disorder and combinations thereof.

According to an embodiment, the primary terpene comprises a dedicated terpene, forming at least 40% by weight of the total amount of said primary terpene. According to an embodiment, the dedicated terpene forms at least 40% by weight of the total amount of said primary terpene, at least 45%, at least 50%, at least 55%, at least 60%, at least 65% or at least 70% of the total amount of said primary terpene.

According to an embodiment, the product comprises at least two primary terpenes or at least one secondary terpene, wherein said therapeutic effect is enhanced compared with that of a product comprising the same amount of cannabinoids and one fifth the amount of said dedicated terpene. According to an embodiment, said therapeutic effect is enhanced compared with that of a product comprising the same amount of cannabinoids and one forth the amount of said dedicated terpene, one third, or one half the amount of said dedicated terpene.

According to an embodiment, the product comprises at least two primary terpenes, and wherein said therapeutic effect is enhanced compared with that of a product comprising the same amount of cannabinoids and one fifth the amount of said dedicated terpene. According to an embodiment, said therapeutic effect is enhanced compared with that of a product comprising the same amount of cannabinoids and one forth the amount of said dedicated terpene, one third, or one half the amount of said dedicated terpene.

According to an embodiment, said dedicated terpene is selected from the group consisting of humulene, caryophyllene, bisbolol, guaiol, pinene linalool, terpineol, limonene, eucalyptol and combinations thereof. According to an embodiment, said dedicated terpene is selected from the group consisting of humulene, caryophyllene, bisbolol and combinations thereof.

According to an embodiment, said dedicated terpene comprises humulene, pinene, caryophyllene, bisabolol, guaiol, limonene and/or eucalyptol, and said product has enhanced therapeutic effect in treating conditions and/or symptoms associated with autism and/or autism spectrum disorder during daytime. According to an embodiment, said dedicated terpene comprises humulene, pinene and/or caryophyllene and said product has enhanced therapeutic effect in treating conditions and/or symptoms associated with autism and/or autism spectrum disorder during daytime.

According to an embodiment, said dedicated terpene comprises caryophyllene, bisabolol, pinene, linalool and/or terpineol and said product has enhanced therapeutic effect in treating conditions and/or symptoms associated with autism and/or autism spectrum disorder during nighttime.

According to an embodiment, provided is a therapeutic kit for daily treating conditions and/or symptoms associated with autism and/or autism spectrum disorder, wherein said dedicated terpene comprises limonene and/or eucalyptol and wherein said dedicated terpene comprises linalool and/or terpineol.

According to an embodiment, said enhanced therapeutic effect comprises a shortened onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof.

According to an embodiment, the primary terpene to cannabinoids weight/weight ratio is greater than 0.05:1. According to an embodiment, the primary terpene to cannabinoids weight/weight ratio is greater than 0.1:1, greater than 0.15:1, greater than 0.2:1, greater than 0.25:1, greater than 0.3:1, greater than 0.35:1, greater than 0.4:1, greater than 0.45:1, greater than 0.5:1, greater than 0.6:1, greater than 0.7:1, greater than 0.8:1, greater than 0.9:1, greater than 1:1, greater than 1.1:1, greater than 1.2:1, greater than 1.3:1 or greater than 1.5:1.

According to an embodiment, the terpenes to cannabinoids weight/weight ratio is greater than 0.05:1. According to an embodiment, the terpenes to cannabinoids weight/weight ratio is greater than 0.1:1, greater than 0.15:1, greater than 0.2:1, greater than 0.25:1, greater than 0.3:1, greater than 0.35:1, greater than 0.4:1, greater than 0.45:1, greater than 0.5:1, greater than 0.6:1, greater than 0.7:1, greater than 0.8:1, greater than 0.9:1, greater than 1:1, greater than 1.1:1, greater than 1.2:1, greater than 1.3:1 or greater than 1.5:1.

According to an embodiment, said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thuj one, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the product comprises less than 5% by weight glycol. According to an embodiment, the product comprises less than 4% by weight glycol, less than 3%, less than 2% or less than 15 glycol. As used herein, the term glycol refers to any glycol, including ethylene glycol, polyethylene glycol, propylene glycol and polypropylene glycol.

According to an embodiment, the product comprises less than 20% by weight water. According to an embodiment, the composition comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5%.

According to an embodiment, the product comprises chlorophyll.

According to an embodiment, the product comprises at least one flavonoid. According to an embodiment, the product comprises at least two flavonoids, at least three, at least four, or at least five.

According to an embodiment, the product comprises at least one of bergamottin, apigenin, amentoflavone, quercetin and piperine.

According to an embodiment, said primary terpene and said cannabinoids are present in specific amounts, and the onset time of said therapeutic effect is at least 20% shorter than that of a product comprising the same cannabinoids amounts and one half the amount of said primary terpene and/or at least 20% shorter than that of a product comprising the same cannabinoids amounts and one half the amount of said dedicated terpene.

According to an embodiment, the primary terpene and the cannabinoids are present in specific amounts, and the magnitude of the therapeutic effect is at least 20% greater compared with that of a product comprising the same cannabinoids amounts and one half the amount of said primary terpene and/or at least 20% greater than that of a product comprising the same cannabinoids amounts and one half the amount of said dedicated terpene.

According to an embodiment, the primary terpene and the cannabinoids are present in specific amounts, and the duration of the therapeutic effect is at least 20% longer compared with that of a product comprising the same cannabinoids amounts and one half the amount of said primary terpene and/or at least 20% longer than that of a product comprising the same cannabinoids amounts and one half the amount of said dedicated terpene.

According to an embodiment, the primary terpene and the cannabinoids are present in specific amounts, and wherein the frequency of said conditions and/or symptoms is at least 20% smaller compared with that of a product comprising the same cannabinoids amounts and one half the amount of said primary terpene and/or at least 20% smaller than that of a product comprising the same cannabinoids amounts and one half the amount of said dedicated terpene.

According to an embodiment, the primary terpene and the cannabinoids are present in specific amounts, and wherein the severity of said conditions and/or symptoms is at least 20% smaller compared with that of a product comprising the same cannabinoids amounts and one half the amount of said primary terpene and/or least 20% smaller than that of a product comprising the same cannabinoids amounts and one half the amount of said dedicated terpene.

According to an embodiment, the primary terpene and the cannabinoids are present in specific amounts, and wherein the consumption of other drugs is reduced by at least 20% compared with that of a product comprising the same cannabinoids amounts and one half the amount of said primary terpene and/or compared with that of a product comprising the same cannabinoids amounts and one half the amount of said dedicated terpene.

According to an embodiment, said product has enhanced therapeutic effect in treating a conditions and/or symptoms associated with autism and/or autism spectrum disorder of a child.

According to an embodiment, said product comprises tetrahydrocannabinol (THC) at a concentration of less than 5% by weight, and at least 0.5% by weight primary terpene.

According to an embodiment, said product has enhanced therapeutic effect in treating conditions and/or symptoms associated with autism and/or autism spectrum disorder of a female.

According to an embodiment, the primary terpene to cannabinoids weight/weight ratio is greater than 0.1:1.

According to an embodiment, the product further comprising a selected amount of herbal preparation, wherein said herbal preparation is produced by at least one of distilling and extracting.

According to an embodiment, said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, lycium barbarum, Matricaria, Melissa officinalis, Mentha piperita, Mitaki, Mumio, Olea europea, origanum, Paeonia lactiflora, panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to an embodiment, the at least one cannabinoid comprises CBD and optionally THC at CBD to THC weight/weight ratio greater than 1:1, wherein said at least one primary terpene is selected from the group consisting of limonene, eucalyptol, linalool, terpineol, caryophyllene, pinene, myrcene, humulene, citronellol, terpinen-4-ol, ocimene, geraniol, menthol, borneol, bisabolol, fenchol and combinations thereof.

According to an embodiment conditions and/or symptoms associated with autism and/or autism spectrum disorder comprise insomnia and/or sleep problems and said at least one cannabinoid comprises at least one of CBD, THC, CBC, CBG, CBN, CBL, THCV and CBDV. According to an embodiment said conditions and/or symptoms associated with autism and/or autism spectrum disorder comprise insomnia and/or sleep problems and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder comprise sleep disorder and/or insomnia and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder comprise insomnia and/or sleep problems and said at least one primary terpene is selected from the group consisting of limonene, eucalyptol, linalool, terpineol, myrcene, caryophyllene, citronellol, pinene, terpinene, nerolidol, sabinene, borneol, fenchol, bisabolol, humulene, phytol, citral and combinations thereof. According to some such embodiments, the at least one primary terpene comprises at least 2 of these terpenes, at least 3, at least 4, or at least 5 of the terpenes. According to an embodiment, the at least one primary terpene is selected from the group consisting of linalool, terpineol, myrcene and combinations thereof. According to an embodiment, the at least one primary terpene is selected from the group consisting of linalool, terpineol, myrcene, caryophyllene, terpinene, borneol and combinations thereof. According to an embodiment, the at least one primary terpene is selected from the group consisting of terpineol, citronellol and linalool and combinations thereof. According to various embodiments said at least one primary terpene is selected from the group consisting of limonene, eucalyptol, linalool, terpineol, myrcene, caryophyllene, citronellol, pinene, terpinene, nerolidol, sabinene, borneol, fenchol, bisabolol, humulene, phytol and citral and combinations thereof. According to an embodiment, said conditions and/or symptoms associated with autism and/or autism spectrum disorder comprise insomnia and/or sleep problems and the product further comprises a compound selected from the group consisting of linalyl acetate and/or chamazulene. According to an embodiment, said conditions and/or symptoms associated with autism and/or autism spectrum disorder comprise insomnia and/or sleep problems and the product comprises an herbal preparation selected from the group consisting of extracts of Mellisa, Bacopa, Valeriane, Avena Sativa, passiflora, Humulus Lopus and combinations thereof. According to an embodiment, said conditions and/or symptoms associated with autism and/or autism spectrum disorder comprise insomnia and/or sleep problems and the product comprises a compound selected from the group consisting of Mirtazapine, melatonin, Donepezil and combinations thereof.

According to an embodiment said conditions and/or symptoms associated with autism and/or autism spectrum disorder are selected from the group consisting of difficulty in concentration, cognitive impairment and attention disorder and combinations thereof, and said at least one cannabinoid is selected from the group consisting of CBD, THC, CBC, CBG, CBN, CBL, THCV and CBDV and combinations thereof. According to an embodiment said conditions and/or symptoms associated with autism and/or autism spectrum disorder are selected from the group consisting of difficulty in concentration, cognitive impairment and attention disorder and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder are selected from the group consisting of difficulty in concentration, cognitive impairment and attention disorder and combinations thereof, and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder are selected from the group consisting of difficulty in concentration, cognitive impairment and/or attention disorder and said at least one primary terpene is selected from the group consisting of limonene, eucalyptol, linalool, terpineol, pinene, pulegone, cymene, ocimene, caryophyllene, humulene, carene myrcene, geraniol, citronellol, menthol, borneol, bisabolol, fenchol and combinations thereof. According to some such embodiments, the product comprises at least 2 of these terpenes, at least 3, at least 4, or at least 5 of the terpenes. According to an embodiment said conditions and/or symptoms associated with autism and/or autism spectrum disorder are selected from the group consisting of difficulty in concentration, cognitive impairment and attention disorder and combinations thereof, and said at least one primary terpene is selected from the group consisting of linalool, eucalyptol, myrcene, and combinations thereof. According to an embodiment, the at least one primary terpene is selected from the group consisting of linalool, eucalyptol, myrcene, caryophyllene, pinene, borneol and combinations thereof. According to an embodiment the at least one primary terpene is selected from the group consisting of limonene, eucalyptol, pinene and combination thereof. According to an embodiment, the at least one primary terpene is selected from the group consisting of linalool, caryophyllene, borneol and combination thereof. According to various embodiments said at least one primary terpene is selected from the group consisting of limonene, eucalyptol, linalool, terpineol, pinene, pulegone, cymene, ocimene, caryophyllene, humulene, carene myrcene, geraniol, citronellol, menthol, borneol, bisabolol and fenchol and combinations thereof. According to an embodiment said conditions and/or symptoms associated with autism and/or autism spectrum disorder are selected from the group consisting of difficulty in concentration, cognitive impairment and attention disorder and combinations thereof, and said product comprises an herbal preparation selected from the group consisting of extracts of Bacopa, Centella, Witania, Rhodiola and combinations thereof. According to an embodiment said conditions and/or symptoms associated with autism and/or autism spectrum disorder are selected from the group consisting of difficulty in concentration, cognitive impairment and attention disorder and combinations thereof, and said product comprises a compound selected from the group consisting of Methylphenidate, Venlafaxine Memantine, rivastigmine and combination thereof.

According to an embodiment said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and depression and combinations thereof, and said at least one cannabnionid is selected from the group consisting of CBD, THC, CBC, CBG, CBN, CBL, THCV and CBDV and combinations thereof. According to an embodiment said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and depression and combinations thereof and said at least one cannabinoid comprises CBD and optionally THC at CBD to THC weight/weight ratio greater than 1:1, greater than 2:1, greater than 3:1, greater than 4:1 or greater than 5:1. According to an embodiment, the at least one cannabinoid comprises CBD and optionally THC at CBD to THC weight/weight ratio greater than 4:1. According to an embodiment said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems, and depression and affect disorder and combinations thereof, and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and depression and combinations thereof, and said at least one primary terpene is selected from the group consisting of limonene, eucalyptol, linalool, terpineol, citronellol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene and combinations thereof. According to some such embodiments, the at least one primary terepene comprises at least 2 of these terpenes, at least 3, at least 4, or at least 5 of the terpenes. According to an embodiment, the at least one primary terpene is selected from the group consisting of limonene, linalool, eucalyptol and combinations thereof. According to an embodiment, the at least one primary terpene is selected from the group consisting of limonene, linalool, eucalyptol, terpineol, caryophyllene, pinene, myrcene and combinations thereof. According to an embodiment, the at least one primary terpene is selected from the group consisting of limonene, linalool, caryophyllene, bisabolol and combinations thereof. According to an embodiment, the at least one primary terpene is selected from the group consisting of limonene, linalool, pinene, humulene and combinations thereof. According to various embodiments said at least one primary terpene is selected from the group consisting of terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol phellandrene and fenchol and combinations thereof. According to an embodiment, said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems andr depression and combinations thereof, and the product further comprises a compound selected from the group consisting of linalyl acetate, geranyl acetate, menthol, piperine, eugenol, citronellyl formate, jasmone, benzy acetate, benzyl alcohol and combinations thereof. According to an embodiment said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and depression and combinations thereof, and said product comprises an herbal preparation selected from the group consisting of extracts of Hypericum Perfortum, Avena Sativa, Passiflora, Humulus Lopus, Centella, Bacopa, mellisa, Verbena officinalis and combinations thereof. According to an embodiment said conditions and/or symptoms associated with autism and/or autism spectrum disorder are selected from the group consisting of anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and r depression and combinations thereof, and said product comprises a compound selected from the group consisting of Risperidone, Aripiprazole, Clozapine, Haloperidol, Sertraline, Oxytocin, Secretin and combinations thereof.

According to an embodiment said conditions and/or symptoms associated with autism and/or autism spectrum disorder are selected from the group consisting of lack of appetite, nausea and vomiting and combinations thereof, and said at least one cannabinoid is selected from the group consisting of CBD, THC, CBC, CBG, CBN, CBL, THCV and CBDV and combinations thereof. According to an embodiment said conditions and/or symptoms associated with autism and/or autism spectrum disorder are selected from the group consisting of lack of appetite, nausea and vomiting and combinations thereof, and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder are selected from the group consisting of lack of appetite, nausea and vomiting and combinations thereof, and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment conditions and/or symptoms associated with autism and/or autism spectrum disorder are selected from the group consisting of lack of appetite, nausea and vomiting and combinations thereof, and said at least one primary terpene is selected from the group consisting of limonene, eucalyptol, linalool, terpineol, citronellol, geraniol, menthol, humulene, bisabolol, borneol, fenchol, pinene, caryophyllene, myrcene, cymene, terpinene and combinations thereof. According to some such embodiments, the at least one primary terepne comprises at least 2 of these terpenes, at least 3, at least 4, or at least 5 of the terpenes. According to an embodiment, the at least one primary terpene is selected from the group consisting of citronellol, linalool caryophyllene and combinations thereof. According to an embodiment, the at least one primary terpene is selected from the group consisting of citronellol, linalool caryophyllene, limonene, myrcene, nerolidol and combinations thereof According to various embodiments said at least one primary terpene is selected from the group consisting of linalool, citronellol, geraniol, menthol, humulene, bisabolol, borneol, fenchol, linalool, limonene, terpineol, pinene, caryophyllene, myrcene, cymene and terpinene and combinations thereof. According to an embodiment said conditions and/or symptoms associated with autism and/or autism spectrum disorder are selected from the group consisting of lack of appetite, nausea and vomiting and combinations thereof, and said product comprises an herbal preparation selected from the group consisting of extracts of Atractylodes macrocephala, Angelica arc, Mentha piperita, Matricaria, Zingiber officinalis and combinations thereof.

According to an embodiment said conditions and/or symptoms associated with autism and/or autism spectrum disorder are selected from the group consisting of digestive problem, incontinence and constipation and combination thereof, and said at least one cannabinoid is selected from the group consisting of CBD, THC, CBC, CBG, CBN, CBL, THCV and CBDV and combinations thereof. According to an embodiment said conditions and/or symptoms associated with autism and/or autism spectrum disorder are selected from the group consisting of digestive problem, incontinence and constipation and combinations thereof, and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder are selected from the group consisting of digestive problem, incontinence and constipation and combinations thereof, and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder are selected from the group consisting of digestive problem, incontinence and constipation and combinations thereof, and said at least one primary terpene is selected from the group consisting of limonene, eucalyptol, linalool, terpineol, cymene, ocimene, terpinene, caryophyllene, citronellol, sabinene, pinene, myrcene, germacrene, geraniol, nerolidol, menthol, humulene, fenchol, borneol and combinations thereof. According to some such embodiments, the at least one primary terpene comprises at least 2 of these terpenes, at least 3, at least 4, or at least 5 of the terpenes. According to an embodiment, the at least one primary terpene is selected from the group consisting of limonene, sabinene, caryophyllene and combinations thereof. According to an embodiment, the at least one primary terpene is selected from the group consisting of limonene, sabinene, caryophyllene, citronellol, linalool, myrcene, nerolidol and combinations thereof According to various embodiments said at least one primary terpene is selected from the group consisting of limonene, cymene, ocimene, terpinene, caryophyllene, citronellol, sabinene, linalool, pinene, myrcene, germacrene, geraniol, nerolidol, menthol, humulene, fenchol and borneol and combinaitons thereof. According to an embodiment, said conditions and/or symptoms associated with autism and/or autism spectrum disorder are selected from the group consisting of digestive problem, incontinence and constipation and combinations thereof, and the product further comprises piperine. According to an embodiment said conditions and/or symptoms associated with autism and/or autism spectrum disorder are selected from the group consisting of digestive problem, incontinence and constipation and combinations thereof, and said product comprises an herbal preparation selected from the group consisting of extracts of Aloe spp, Angelica arc, Rumex, Silybum marianum, Rehmania glutinosa, Taraxacum, Humulus lupus, Verbena officinalis and combinations thereof.

According to an embodiment said conditions and/or symptoms associated with autism and/or autism spectrum disorder are selected from the group consisting of stereotypy, inappropriate speech, motor tics and vocal tics and combinations thereof, and said at least one cannabinoid is selected from the group consisting of CBD, THC, CBC, CBG, CBN, CBL, THCV and CBDV and combinations thereofs. According to an embodiment said conditions and/or symptoms associated with autism and/or autism spectrum disorder are selected from the group consisting of stereotypy, inappropriate speech, motor tics and vocal tics and combinations thereof, and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder are selected from the group consisting of stereotypy, inappropriate speech, motor tics and vocal tics and combinations thereof, and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder are selected from the group consisting of stereotypy, inappropriate speech, motor tics and vocal tics and combinations thereof, and said at least one primary terpene is selected from the group consisting of limonene, eucalyptol, linalool, terpineol, myrcene, pinene, nerolidol, caryophyllene, geraniol, menthol, borneol, bisabolol, fenchol and combinations thereof. According to some such embodiments, the at least one primary terpene comprises at least 2 of these terpenes, at least 3, at least 4, or at least 5 of the terpenes. According to an embodiment, the at least one primary terpene is selected from the group consisting of myrcene, limonene, nerolidol and combinations thereof. According to an embodiment, the primary terpene is selected from the group consisting of linalool, pinene, eucalyptol and combinations thereof. According to various embodiments said at least one primary terpene is selected from the group consisting of myrcene, linalool, limonene, pinene, nerolidol, caryophyllene, eucalyptol, geraniol, menthol, borneol, bisabolol and fenchol and combinations thereof. According to an embodiment said conditions and/or symptoms associated with autism and/or autism spectrum disorder are selected from the group consisting of stereotypy, inappropriate speech, motor tics and vocal tics and combinations thereof, and said product comprises an herbal preparation selected from the group consisting of extracts of Cypripedium pubescens, whitania, Hypericum, Rhodiola, Vallerian, passiflora, Bacopa, Centella, Rhodiola and combinations thereof. According to an embodiment said conditions and/or symptoms associated with autism and/or autism spectrum disorder are selected from the group consisting of stereotypy, inappropriate speech, motor tics andr vocal tics and combinations thereof, and said administered product comprises a compound selected from the group consisting of Fluoxetine, Citalopram, Bumetanide and combinations thereof.

According to an embodiment said conditions and/or symptoms associated with autism and/or autism spectrum disorder comprise seizure activity and said at least one cannabinoid is selected from the group consisting of CBD, THC, CBC, CBG, CBN, CBL, THCV and CBDV and combinations thereof. According to an embodiment conditions and/or symptoms associated with autism and/or autism spectrum disorder comprise seizure activity and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder comprise seizure activity and said at least one cannabinoid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder comprise seizure activity and said at least one primary terpene is selected from the group consisting of limonene, eucalyptol, linalool, terpineol ocimene, myrcene, limonene, caryophyllene and combinations thereof. According to some such embodiments, the at last one primary terpene comprises at least 2 of these terpenes, at least 3, at least 4, or at least 5 of the terpenes. According to an embodiment, the at least one primary terpene is selected from the group consisting of linalool, terpineol, ocimene and combinations thereof. According to various embodiments said at least one primary terpene is selected from the group consisting of linalool, terpineol, ocimene, myrcene, limonene and caryophyllene and combinations thereof. According to an embodiment said conditions and/or symptoms associated with autism and/or autism spectrum disorder comprise seizure activity and said product comprises an herbal preparation selected from the group consisting of extracts of Bacopa, centella, Curcuma, Whitania, Zingiber officinalis, Peonia and combinations thereof.

According to an embodiment said conditions and/or symptoms associated with autism and/or autism spectrum disorder are selected from the group consisting of lethargy, fatigue and deficits in balance and combinations thereof, and said at least one cannabinnoid is selected from the group consisting of CBD, THC, CBC, CBG, CBN, CBL, THCV and CBDV and combinations thereof. According to an embodiment said conditions and/or symptoms associated with autism and/or autism spectrum disorder are selected from the group consisting of lethargy, fatigue and deficits in balance and combinations thereof, and said at least one cannabinoid comprises CBD and optionally THC at CBD to THC weight/weight ratio greater than 1:1, greater than 2:1, greater than 3:1, greater than 4:1 or greater than 5:1. According to an embodiment said conditions and/or symptoms associated with autism and/or autism spectrum disorder are selected from the group consisting of lethargy, fatigue and deficits in balance and combinations thereof, and said at least one cannabinonid comprises THC and optionally CBD at THC to CBD weight/weight ratio greater than 0.9:1, greater than 2:1, greater than 3:1, greater than 4:1 or greater than 5:1. According to various embodiments, said at least one cannabinoid comprises CBD, comprises THC, comprises CBC, comprises CBG, comprises CBN, comprises CBL, comprises THCV and/or comprises CBDV. According to an embodiment said conditions and/or symptoms associated with autism and/or autism spectrum disorder are selected from the group consisting of lethargy, fatigue and/or deficits in balance and said the primary terpene is selected from the group consisting of limonene, eucalyptol, linalool, terpineol pinene, terpinene, myrcene, caryophyllene, borneo and combinations thereof. According to some such embodiments, the at least one primary terpene comprises at least 2 of these terpenes, at least 3, at least 4, or at least 5 of the terpenes. According to an embodiment, the at least one primary terpene is selected from the group consisting of pinene, terpinene, eucalyptol and combinations thereof. According to an embodiment, the at least one primary terpene is selected from the group consisting of limonene, myrcene, borneol and combinations thereof. According to various embodiments said at least one primary terpene is selected from the group consisting of terpinene, myrcene, borneol and caryophyllene and combinations thereof. According to an embodiment said conditions and/or symptoms associated with autism and/or autism spectrum disorder are selected from the group consisting of lethargy, fatigue and deficits in balance and combinations thereof, and said product comprises an herbal preparation selected from the group consisting of extracts of centella, Rhodiola, Whitania, Astragalus membranous, Bacopa, Ganoderma and combinations thereof.

According to an embodiment, said product comprises a compound selected from the group consisting of Mirtazapine, melatonin, Donepezil, Methylphenidate, Venlafaxine Memantine, rivastigmine, Risperidone, Aripiprazole, Clozapine, Haloperidol, Sertraline, Oxytocin, Secretin, Fluoxetine, Citalopram, Bumetanide, Acamprosate, Atomoxetine, baclofen, DMXB-A (a derivative of the nemertine toxin anabasein), EPI-743 (vincerinone), RG7314 (Roche), Divalproex, Dextromethorphan hydrobromide, and/or quinidine sulfate and combinations thereof.

According to an embodiment, the product is provided in a form selected from the group consisting of medical patches, stickers, topicals, creams, varnishes, sprays, edibles, beverages, suppositories, nasal preparations, inhalation mask, sub-lingual tabs, lozenges, chewing gums, preparations containing micro and/or nano-emulsions, preparations containing micro and/or nano-particles and combinations thereof. According to an embodiment, provided is a method of treating conditions and/or symptoms associated with autism and/or autism spectrum disorder, the method comprising administering to a subject in need thereof a therapeutically effective amount of a product comprising (i) at least one cannabinoid in a specific amount, (ii) at least one primary terpene in a specific amount, and (iii) optionally at least three secondary terpenes, wherein said primary terpene forms at least 20% of the total amount of terpenes in said product. According to an embodiment, the product has an enhanced therapeutic effect in treating conditions and/or symptoms associated with autism and/or autism spectrum disorder, compared with administrating a product comprising the same amount of cannabinoids and one fifth the amount of said at least one primary terpene.

According to an aspect of some embodiment of the invention, there is provided a method of treating conditions and/or symptoms associated with autism and/or autism spectrum disorder comprising administering to a subject in need thereof a therapeutically effective amount of a product comprising

(i) at least one cannabinoid,
(ii) at least one primary terpene, and
(iii) optionally at least three secondary terpenes,
wherein said at least one primary terpene forms at least 20% of the total amount of terpenes in said product.

According to an aspect of some embodiment of the invention, there is provided a product comprising

(i) at least one cannabinoid,
(ii) at least one primary terpene, and
(iii) optionally at least three secondary terpenes,
wherein said at least one primary terpene forms at least 20% of the total amount of terpenes in said product,
for use in the treatment of conditions and/or symptoms associated with autism and/or autism spectrum disorder.
According to an embodiment, the product is a composition.

As used herein, the term “treating” includes preventing, curing, ameliorating, mitigating, and reducing the instances or severity of a condition or a symptom thereof.

According to an embodiment, said conditions and/or symptoms associated with autism and/or autism spectrum disorder are selected from the group consisting of of restlessness, rage attack, agitation, treatment-resistant self-injurious behavior, stress, aggression, anxiety, irritability, behavioral outbreaks, communication problems, disruptive behavior, psychosis, hyperactivity, lethargy, seizure activity, hyper sensitivity, stereotypy, inappropriate speech, sleep problem, cognitive impairment, motor and/or vocal tics, digestive problems, lack of appetite, constipation, depression, incontinence, difficulty in concentration, attention disorder and combinations thereof.

According to an embodiment, said at least one primary terpene comprises a dedicated terpene, forming at least 40% by weight of the total amount of said primary terpenes.

According to an embodiment, said product comprises multiple terpenes, wherein the multiple terpenes comprise at least two primary terpenes or at least one primary terpene and at least one secondary terpene, and said therapeutic effect is enhanced compared with that of a product comprising the same amount of cannabinoids and one fifth the amount of said dedicated terpene.

According to an embodiment, said at least one primary terpene comprises multiple terpenes i.e. at least two primary terpenes, and said therapeutic effect is enhanced compared with that of a product comprising the same amount of cannabinoids and one fifth the amount of said dedicated terpene.

According to an embodiment, said dedicated terpene is selected from the group consisting of humulene, caryophyllene, bisbolol, guaiol, pinene linalool, terpineol, limonene, eucalyptol and combinations thereof. According to an embodiment, said dedicated terpene is selected from the group consisting of humulene, caryophyllene, bisbolol and combinations thereof.

According to an embodiment, said dedicated terpene comprises humulene, pinene, caryophyllene, bisabolol, guaiol, limonene and/or eucalyptol, and wherein said product has enhanced therapeutic effect in treating conditions and/or symptoms associated with autism and/or autism spectrum disorder during daytime. According to an embodiment, said dedicated terpene comprises humulene, pinene and/or caryophyllene and said product has enhanced therapeutic effect in treating conditions and/or symptoms associated with autism and/or autism spectrum disorder during daytime.

According to an embodiment, said method is used to alleviate daytime adverse behaviors, including behavioral outbreaks, aggressive behavior, rage attacks, self-injurious behavior, irritability, restlessness, disruptive behavior, psychosis, hyperactivity, hyper sensitivity, stereotypy, inappropriate speech, and/or motor or vocal tics, and to improve communication, attention, learning capabilities and daily functioning.

According to an embodiment, said dedicated terpene comprises caryophyllene, bisabolol, pinene, linalool and/or terpineol and wherein said product has enhanced therapeutic effect in treating conditions and/or symptoms associated with autism and/or autism spectrum disorder during nighttime.

According to an embodiment, said method is used to improve sleep and/or to improve calmness and/or relaxation.

According to an embodiment, provided is a daily method for treating conditions and/or symptoms associated with autism and/or autism spectrum disorder, wherein said dedicated terpene comprises limonene and/or eucalyptol and wherein said dedicated terpene comprises linalool and/or terpineol.

According to an embodiment, said method is used to treat a subject, wherein said subject is an autistic disorder subject.

According to an embodiment, said method is used to treat a subject, wherein said subject is an Asperger's syndrome subject. According to an embodiment, said method is used for treating conditions and/or symptoms associated with autism and/or autism spectrum disorder comprise Asperger's syndrome.

According to an embodiment, said method is used to treat a subject, wherein said subject is a pervasive developmental disorder not otherwise specified (PDD-NOS) subject. According to an embodiment, said method is used for treating conditions and/or symptoms associated with autism and/or autism spectrum disorder comprise pervasive developmental disorder not otherwise specified (PDD-NOS).

According to an embodiment, said method is used to treat a subject, wherein said subject is a Rett syndrome subject. According to an embodiment, said method is used for treating conditions and/or symptoms associated with autism and/or autism spectrum disorder comprise Rett syndrome.

According to an embodiment, said method is used to treat a subject, wherein said subject has an intellectual disability. According to an embodiment, said method is used to treat a subject, wherein said subject has no intellectual disability.

According to an embodiment, said method is used to treat conditions and/or symptoms associated with autism and/or autism spectrum disorder, wherein said conditions and/or symptoms comprise severe symptoms. According to an embodiment, said method is used to treat conditions and/or symptoms associated with autism and/or autism spectrum disorder, wherein said conditions and/or symptoms comprise mild symptoms.

According to an embodiment, said method is used to treat conditions and/or symptoms associated with autism and/or autism spectrum disorder, wherein said autism and/or autism spectrum disorder is primarily characterized by language impartments.

According to an embodiment, said method is used to treat conditions and/or symptoms associated with autism and/or autism spectrum disorder, wherein said autism and/or autism spectrum disorder is primarily characterized by social and communication disabilities.

According to an embodiment, said method is used to treat conditions and/or symptoms associated with autism and/or autism spectrum disorder, wherein said autism and/or autism spectrum disorder is primarily characterized by unusual behaviors and interests.

According to an embodiment, said method is used to treat conditions and/or symptoms associated with autism and/or autism spectrum disorder, wherein said administrated product is administrated during morning time. According to an embodiment, said administrated product is administrated during noon time. According to an embodiment, said administrated product is administrated during evening time. According to an embodiment, said administrated product is administrated during night time.

According to an embodiment, said method is used to treat conditions and/or symptoms associated with autism and/or autism spectrum disorder, wherein said administrated product is administrated once a day. According to an embodiment, said administrated product is administrated twice a day, at least three times a day, at least four time a days, or at least five time a day.

According to an embodiment, said method is used to treat conditions and/or symptoms associated with autism and/or autism spectrum disorder, wherein said administrated product is implemented within at least one of milk products, choloclate products, honey products and fruits.

According to an embodiment, said enhanced therapeutic effect comprises a shortened onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof.

According to an embodiment, the primary terpene to cannabinoids weight/weight ratio is greater than 0.05:1.

According to an embodiment, the terpenes to cannabinoids weight/weight ratio is greater than 0.05:1.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thuj one, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the administrated product comprises less than 5% by weight glycol.

According to an embodiment, the administrated product comprises less than 20% by weight water.

According to an embodiment, the administrated product comprises chlorophyll.

According to an embodiment, the administrated product comprises at least one flavonoid.

According to an embodiment, the administrated product comprises at least one of bergamottin, apigenin, amentoflavone, quercetin and piperine.

According to an embodiment, said primary terpene and said cannabinoids are present in specific amounts, and the onset time of said therapeutic effect is at least 20% shorter than that of a product comprising the same cannabinoids amounts and one half the amount of said primary terpene and/or at least 20% shorter than that of a product comprising the same cannabinoids amounts and one half the amount of said dedicated terpene.

According to an embodiment, the primary terpene and the cannabinoids are present in specific amounts, and the magnitude of the therapeutic effect is at least 20% greater compared with that of a product comprising the same cannabinoids amounts and one half the amount of said primary terpene and/or at least 20% greater than that of a product comprising the same cannabinoids amounts and one half the amount of said dedicated terpene.

According to an embodiment, the primary terpene and the cannabinoids are present in specific amounts, and the duration of the therapeutic effect is at least 20% longer compared with that of a product comprising the same cannabinoids amounts and one half the amount of said primary terpene and/or at least 20% longer than that of a product comprising the same cannabinoids amounts and one half the amount of said dedicated terpene.

According to an embodiment, the primary terpene and the cannabinoids are present in specific amounts, and wherein the frequency of said conditions and/or symptoms is at least 20% smaller compared with that of a product comprising the same cannabinoids amounts and one half the amount of said primary terpene and/or at least 20% smaller than that of a product comprising the same cannabinoids amounts and one half the amount of said dedicated terpene.

According to an embodiment, the primary terpene and the cannabinoids are present in specific amounts, and wherein the severity of said conditions and/or symptoms is at least 20% smaller compared with that of a product comprising the same cannabinoids amounts and one half the amount of said primary terpene and/or least 20% smaller than that of a product comprising the same cannabinoids amounts and one half the amount of said dedicated terpene.

According to an embodiment, the primary terpene and the cannabinoids are present in specific amounts, and wherein the consumption of other drugs is reduced by at least 20% compared with that of a product comprising the same cannabinoids amounts and one half the amount of said primary terpene and/or compared with that of a product comprising the same cannabinoids amounts and one half the amount of said dedicated terpene.

According to an embodiment, said product has enhanced therapeutic effect in treating a conditions and/or symptoms associated with autism and/or autism spectrum disorder of a child.

According to an embodiment, said product comprises tetrahydrocannabinol (THC) at a concentration of less than 5% by weight, and at least 0.5% by weight primary terpene.

According to an embodiment, said product has enhanced therapeutic effect in treating conditions and/or symptoms associated with autism and/or autism spectrum disorder of a female.

According to an embodiment, the primary terpene to cannabinoids weight/weight ratio is greater than 0.1:1.

According to an embodiment, the product further comprising a selected amount of herbal preparation, wherein said herbal preparation is produced by at least one of distilling and extracting.

According to an embodiment, said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, lycium barbarum, Matricaria, Melissa officinalis, Mentha piperita, Mitaki, Mumio, Olea europea, origanum, Paeonia lactiflora, panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to an embodiment, said administrated product comprises CBD and optionally THC at CBD to THC weight/weight ratio greater than 1:1, wherein said primary terpene is selected from the group consisting of limonene, eucalyptol, linalool, terpineol, caryophyllene, pinene, myrcene, humulene, citronellol, terpinen-4-ol, ocimene, geraniol, menthol, borneol, bisabolol, fenchol and combinations thereof.

According to an embodiment, said primary terpene comprises a dedicated terpene.

According to an embodiment said therapeutic effect treats sleep insomnia and/or sleep problems and said product comprises at least one of CBD, THC, CBC, CBG, CBN, CBL, THCV and CBDV. According to an embodiment said therapeutic effect treats insomnia and/or sleep problems and said product comprises CBD and optionally THC at CBD to THC weight/weight ratio greater than 1, greater than 2, greater than 3, greater than 4 or greater than 5. According to an embodiment said therapeutic effect treats sleep disorder and/or insomnia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats insomnia and/or sleep problems and said the primary terpene is selected from the group consisting of limonene, eucalyptol, linalool, terpineol, myrcene, caryophyllene, citronellol, pinene, terpinene, nerolidol, sabinene, borneol, fenchol, bisabolol, humulene, phytol, citral and combinations thereof. According to an embodiment, the product comprises at least 2 of these terpenes, at least 3, at least 4, or at least 5 of the terpenes. According to an embodiment, the primary terpene is selected from the group consisting of linalool, terpineol, myrcene and combinations thereof. According to an embodiment, the primary terpene is selected from the group consisting of linalool, terpineol, myrcene, caryophyllene, terpinene, borneol and combinations thereof. According to an embodiment, the primary terpene comprises terpineol, citronellol and/or linalool. According to various embodiments said primary terpene comprises limonene, eucalyptol, linalool, terpineol, myrcene, caryophyllene, citronellol, pinene, terpinene, nerolidol, sabinene, borneol, fenchol, bisabolol, humulene, phytol and/or citral. According to an embodiment, said therapeutic effect treats insomnia and/or sleep problems and the product further comprises a compound selected from the group consisting of linalyl acetate and/or chamazulene. According to an embodiment, said therapeutic effect treats insomnia and/or sleep problems and the product comprises an herbal preparation selected from the group consisting of extracts of Mellisa, Bacopa, Valeriane, Avena Sativa, passiflora, Humulus Lopus and combinations thereof. According to an embodiment, said therapeutic effect treats insomnia and/or sleep problems and the product comprises a compound selected from the group consisting of Mirtazapine, melatonin, Donepezil and combinations thereof.

According to an embodiment said therapeutic effect treats difficulty in concentration, cognitive impairment and/or attention disorder and said product comprises at least one of CBD, THC, CBC, CBG, CBN, CBL, THCV and CBDV. According to an embodiment said therapeutic effect treats difficulty in concentration, cognitive impairment and/or attention disorder and said product comprises CBD and optionally THC at CBD to THC weight/weight ratio greater than 1, greater than 2, greater than 3, greater than 4 or greater than 5. According to an embodiment said therapeutic effect treats difficulty in concentration, cognitive impairment and/or attention disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats difficulty in concentration, cognitive impairment and/or attention disorder and said primary terpene is selected from the group consisting of limonene, eucalyptol, linalool, terpineol, pinene, pulegone, cymene, ocimene, caryophyllene, humulene, carene myrcene, geraniol, citronellol, menthol, borneol, bisabolol, fenchol and combinations thereof. According to an embodiment, the product comprises at least 2 of these terpenes, at least 3, at least 4, or at least 5 of the terpenes. According to an embodiment said therapeutic effect treats difficulty in concentration, cognitive impairment and/or attention disorder and said primary terpene is selected from the group consisting of linalool, eucalyptol, myrcene, and combinations thereof. According to an embodiment, the primary terpene is selected from the group consisting of linalool, eucalyptol, myrcene, caryophyllene, pinene, borneol and combinations thereof. According to an embodiment the primary terpene is selected from the group consisting of limonene, eucalyptol, pinene and combination thereof. According to an embodiment, the primary terpene is selected from the group consisting of linalool, caryophyllene, borneol and combination thereof. According to various embodiments said primary terpene comprises limonene, eucalyptol, linalool, terpineol, pinene, pulegone, cymene, ocimene, caryophyllene, humulene, carene myrcene, geraniol, citronellol, menthol, borneol, bisabolol and/or fenchol. According to an embodiment said therapeutic effect treats difficulty in concentration, cognitive impairment and/or attention disorder and said product comprises an herbal preparation selected from the group consisting of extracts of Bacopa, Centella, Witania, Rhodiola and combinations thereof. According to an embodiment said therapeutic effect treats difficulty in concentration, cognitive impairment and/or attention disorder and said product comprises a compound selected from the group consisting of Methylphenidate, Venlafaxine Memantine, rivastigmine and combination thereof.

According to an embodiment said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and said product comprises at least one of CBD, THC, CBC, CBG, CBN, CBL, THCV and CBDV. According to an embodiment said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and said product comprises CBD and optionally THC at CBD to THC weight/weight ratio greater than 1, greater than 2, greater than 3, greater than 4 or greater than 5. According to an embodiment, the product comprises CBD and optionally THC at CBD to THC weight/weight ratio greater than 4:1. According to an embodiment said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and/or affect disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and said the primary terpene is selected from the group consisting of limonene, eucalyptol, linalool, terpineol, citronellol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene and combinations thereof. According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, eucalyptol, terpineol, caryophyllene, pinene, myrcene and combinations thereof. According to an embodiment, the primary terpene is selected from the group consisting of limonene, linalool, caryophyllene, bisabolol and combinations thereof. According to an embodiment, the primary terpene is selected from the group consisting of limonene, linalool, pinene, humulene and combinations thereof. According to various embodiments said primary terpene comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol phellandrene and/or fenchol. According to an embodiment, said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and the product further comprises a compound selected from the group consisting of linalyl acetate, geranyl acetate, menthol, piperine, eugenol, citronellyl formate, jasmone, benzy acetate, benzyl alcohol and combinations thereof. According to an embodiment said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and said product comprises an herbal preparation selected from the group consisting of extracts of Hypericum Perfortum, Avena Sativa, Passiflora, Humulus Lopus, Centella, Bacopa, mellisa, verbena officinalis and combinations thereof. According to an embodiment said therapeutic effect treats anxiety, irritability, nervousness, restlessness, stress, aggression, hyperactivity, disruptive behavior, psychosis, behavioral outbreaks, hyper sensitivity, rage attack, agitation, treatment-resistant self-injurious behavior, communication problems and/or depression and said product comprises a compound selected from the group consisting of Risperidone, Aripiprazole, Clozapine, Haloperidol, Sertraline, Oxytocin, Secretin and combinations thereof.

According to an embodiment said therapeutic effect treats lack lack of appetite, nausea and or/vomiting and said product comprises at least one of CBD, THC, CBC, CBG, CBN, CBL, THCV and CBDV. According to an embodiment said therapeutic effect treats lack of appetite, nausea and or/vomiting and said product comprises CBD and optionally THC at CBD to THC weight/weight ratio greater than 1, greater than 2, greater than 3, greater than 4 or greater than 5. According to an embodiment said therapeutic effect treats lack of appetite, nausea and or/vomiting and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats lack of appetite, nausea and or/vomiting and said the primary terpene is selected from the group consisting of limonene, eucalyptol, linalool, terpineol, citronellol, geraniol, menthol, humulene, bisabolol, borneol, fenchol, pinene, caryophyllene, myrcene, cymene, terpinene and combinations thereof. According to an embodiment, the product comprises at least 2 of these terpenes, at least 3, at least 4, or at least 5 of the terpenes. According to an embodiment, the primary terpene is selected from the group consisting of citronellol, linalool caryophyllene and combinations thereof. According to an embodiment, the primary terpene is selected from the group consisting of citronellol, linalool caryophyllene, limonene, myrcene, nerolidol and combinations thereof. According to various embodiments said primary terpene comprises linalool, citronellol, geraniol, menthol, humulene, bisabolol, borneol, fenchol, linalool, limonene, terpineol, pinene, caryophyllene, myrcene, cymene and/or terpinene. According to an embodiment said therapeutic effect treats lack lack of appetite, nausea and or/vomiting and said product comprises an herbal preparation selected from the group consisting of extracts of Atractylodes macrocephala, Angelica arc, Mentha piperita, Matricaria, Zingiber officinalis and combinations thereof.

According to an embodiment said therapeutic effect treats digestive problem, incontinence and/or constipation and said product comprises at least one of CBD, THC, CBC, CBG, CBN, CBL, THCV and CBDV. According to an embodiment said therapeutic effect treats digestive problem, incontinence and/or constipation and said product comprises CBD and optionally THC at CBD to THC weight/weight ratio greater than 1, greater than 2, greater than 3, greater than 4 or greater than 5. According to an embodiment said therapeutic effect treats digestive problem, incontinence and/or constipation and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats digestive problem, incontinence and/or constipation and said the primary terpene is selected from the group consisting of limonene, eucalyptol, linalool, terpineol, cymene, ocimene, terpinene, caryophyllene, citronellol, sabinene, pinene, myrcene, germacrene, geraniol, nerolidol, menthol, humulene, fenchol, borneol and combinations thereof. According to an embodiment, the product comprises at least 2 of these terpenes, at least 3, at least 4, or at least 5 of the terpenes. According to an embodiment, the primary terpene is selected from the group consisting of limonene, sabinene, caryophyllene and combinations thereof. According to an embodiment, the primary terpene is selected from the group consisting of limonene, sabinene, caryophyllene, citronellol, linalool, myrcene, nerolidol and combinations thereof. According to various embodiments said primary terpene comprises limonene, cymene, ocimene, terpinene, caryophyllene, citronellol, sabinene, linalool, pinene, myrcene, germacrene, geraniol, nerolidol, menthol, humulene, fenchol and/or borneol. According to an embodiment, said therapeutic effect treats digestive problem, incontinence and/or constipation and the product further comprises piperine. According to an embodiment said therapeutic effect treats digestive problem, incontinence and/or constipation and said product comprises an herbal preparation selected from the group consisting of extracts of Aloe spp, Angelica arc, Rumex, Silybum marianum, Rehmania glutinosa, Taraxacum, Humulus lupus, Verbena officinalis and combinations thereof.

According to an embodiment said therapeutic effect treats stereotypy, inappropriate speech, motor tics and/or vocal tics and said administered product comprises at least one of CBD, THC, CBC, CBG, CBN, CBL, THCV and CBDV. According to an embodiment said therapeutic effect treats stereotypy, inappropriate speech, motor tics and/or vocal tics and said product comprises CBD and optionally THC at CBD to THC weight/weight ratio greater than 1, greater than 2, greater than 3, greater than 4 or greater than 5. According to an embodiment said therapeutic effect treats stereotypy, inappropriate speech, motor tics and/or vocal tics and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats stereotypy, inappropriate speech, motor tics and/or vocal tics and said primary terpene is selected from the group consisting of limonene, eucalyptol, linalool, terpineol, myrcene, pinene, nerolidol, caryophyllene, geraniol, menthol, borneol, bisabolol, fenchol and combinations thereof. According to an embodiment, the product comprises at least 2 of these terpenes, at least 3, at least 4, or at least 5 of the terpenes. According to an embodiment, the primary terpene is selected from the group consisting of myrcene, limonene, nerolidol and combinations thereof. According to an embodiment, the primary terpene is selected from the group consisting of linalool, pinene, eucalyptol and combinations thereof. According to various embodiments said primary terpene comprises myrcene, linalool, limonene, pinene, nerolidol, caryophyllene, eucalyptol, geraniol, menthol, borneol, bisabolol and/or fenchol. According to an embodiment said therapeutic effect treats stereotypy, inappropriate speech, motor tics and/or vocal tics and said administered product comprises an herbal preparation selected from the group consisting of extracts of Cypripedium pubescens, whitania, Hypericum, Rhodiola, Vallerian, passiflora, Bacopa, Centella, Rhodiola and combinations thereof. According to an embodiment said therapeutic effect treats stereotypy, inappropriate speech, motor tics and/or vocal tics and said administered product comprises a compound selected from the group consisting of Fluoxetine, Citalopram, Bumetanide and combinations thereof.

According to an embodiment said therapeutic effect treats seizure activity and said administered product comprises at least one of CBD, THC, CBC, CBG, CBN, CBL, THCV and CBDV. According to an embodiment said therapeutic effect treats seizure activity and said product comprises CBD and optionally THC at CBD to THC weight/weight ratio greater than 1, greater than 2, greater than 3, greater than 4 or greater than 5. According to an embodiment said therapeutic effect treats seizure activity and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats seizure activity and said primary terpene is selected from the group consisting of limonene, eucalyptol, linalool, terpineol ocimene, myrcene, limonene, caryophyllene and combinations thereof. According to an embodiment, the product comprises at least 2 of these terpenes, at least 3, at least 4, or at least 5 of the terpenes. According to an embodiment, the primary terpene is selected from the group consisting of linalool, terpineol, ocimene and combinations thereof. According to various embodiments said primary terpene comprises linalool, terpineol, ocimene, myrcene, limonene and/or caryophyllene. According to an embodiment said therapeutic effect treats seizure activity and said administered product comprises an herbal preparation selected from the group consisting of extracts of Bacopa, centella, Curcuma, Whitania, Zingiber officinalis, Peonia and combinations thereof.

According to an embodiment said therapeutic effect treats lethargy, fatigue and/or deficits in balance and said administered product comprises at least one of CBD, THC, CBC, CBG, CBN, CBL, THCV and CBDV. According to an embodiment said therapeutic effect treats lethargy, fatigue and/or deficits in balance and said product comprises CBD and optionally THC at CBD to THC weight/weight ratio greater than 1, greater than 2, greater than 3, greater than 4 or greater than 5. According to an embodiment said therapeutic effect treats lethargy, fatigue and/or deficits in balance and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats lethargy, fatigue and/or deficits in balance and said the primary terpene is selected from the group consisting of limonene, eucalyptol, linalool, terpineol pinene, terpinene, myrcene, caryophyllene, borneo and combinations thereof. According to an embodiment, the product comprises at least 2 of these terpenes, at least 3, at least 4, or at least 5 of the terpenes. According to an embodiment, the primary terpene is selected from the group consisting of pinene, terpinene, eucalyptol and combinations thereof. According to an embodiment, the primary terpene is selected from the group consisting of limonene, myrcene, borneol and combinations thereof. According to various embodiments said primary terpene comprises terpinene, myrcene, borneol and/or caryophyllene. According to an embodiment said therapeutic effect treats lethargy, fatigue and/or deficits in balance and said administered product comprises an herbal preparation selected from the group consisting of extracts of centella, Rhodiola, Whitania, Astragalus membranous, Bacopa, Ganoderma and combinations thereof.

According to an embodiment, said administered product comprises a compound selected from the group consisting of Mirtazapine, melatonin, Donepezil, Methylphenidate, Venlafaxine Memantine, rivastigmine, Risperidone, Aripiprazole, Clozapine, Haloperidol, Sertraline, Oxytocin, Secretin, Fluoxetine, Citalopram, Bumetanide, Acamprosate, Atomoxetine, baclofen, DMXB-A (a derivative of the nemertine toxin anabasein), EPI-743 (vincerinone), RG7314 (Roche), Divalproex, Dextromethorphan hydrobromide, and/or quinidine sulfate and combinations thereof.

According to an embodiment, said administrated product is selected from the group consisting of medical patches, stickers, topicals, creams, varnishes, sprays, edibles, beverages, suppositories, nasal preparations, inhalation mask, sub-lingual tabs, lozenges, chewing gums, preparations containing micro and/or nano-emulsions, preparations containing micro and/or nano-particles and combinations thereof.

### Examples 1-38

The table below presents examples of compositions as described herein according to the following legend:

| | A | B | C | D | E | F | G |
|---|---|---|---|---|---|---|---|
| 1 | Oil | CBD | 5 | Caryophyllene | Caryophyllene | Bacopa | Attention |
| | | (20) | | (3) | (3) | | disorder |
| | | | | Limonene (1) | | | |
| | | | | Eucalyptol (1) | | | |
| 2 | Oil | THC | 3.1 | Linalool (3) | Linalool (3) | | Nighttime |
| | | (10) | | | | | composition |
| 3 | Oil | THC | 5.2 | Nerolidol (1) | Limonene (3) | Rhodiola | vocal tics |
| | | (18) | | Limonene (3) | | | |
| | | | | linalool (1) | | | |
| 4 | Oil | CBD | 5 | Caryophyllene | Caryophyllene | | Difficulty in |
| | | (20) | | (3) | (3) | | concentration |
| | | | | pulegone (1) | | | |
| | | | | terpineol (1) | | | |
| 5 | Oil | THC | 4.2 | Limonene (2) | Limonene (2) | Hypericum | aggression |
| | | and | | Eucalyptol (1) | | Perfortum | |
| | | CBD | | Linalool (1) | | | |
| | | (20) | | | | | |
| 6 | Oil | THC | 3 | Caryophyllene | Caryophyllene | Avena Sativa | hyperactivity |
| | | and | | (2) | (2) | | |
| | | CBD | | Geraniol (0.5) | | | |
| | | (18) | | Menthol (0.5) | | | |
| 7 | Oil | THC | 4.1 | Limonene (2) | Limonene (2) | | digestive |
| | | and | | Menthol (1) | | | problems |
| | | CBD | | Geraniol (1) | | | |
| | | (18) | | | | | |
| 8 | Oil | CBD | 3 | Eucalyptul (1) | Limonene (2) | Ganoderma | lethargy |
| | | (12) | | Limonene (2) | | | |
| 9 | Oil | CBD | 5 | Caryophyllene | Limonene (3) | | Daytime |
| | | (20) | | (1) | | | composition |
| | | | | Limonene (3) | | | |
| | | | | Eucalyptol (1) | | | |
| 10 | Oil | THC | 4.2 | Bisabolol (4) | Bisabolol (4) | Humulus Lopus | insomnia |
| | | (24) | | | | | |
| 11 | Oil | CBD | 3.1 | Linalool (3) | Linalool (3) | | stress |
| | | (15) | | | | | |
| 12 | Oil | CBD | 4.2 | Eucalyptol (3) | Eucalyptol (3) | | attention |
| | | (12) | | Linalool (1) | | | disorder |
| 13 | Oil | THC | 1 | Linalool (1) | Linalool (1) | mellisa | restlessness |
| | | (5) | | | | | |
| 14 | Oil | THC | 5.2 | Terpineol (3) | Terpineol (3) | | agitation |
| | | and | | Eucalyptol (2) | | | |
| | | CBD | | | | | |
| | | (15) | | | | | |
| 15 | Oil | CBD | 5.2 | Caryophyllene | Caryophyllene | | Daytime |
| | | (20) | | (3) | (3) | | composition |
| | | | | Limonene (1) | | | |
| | | | | Eucalyptol (1) | | | |
| 16 | Oil | CBD | 2 | Eucalyptol (0.5) | Linalool (1.5) | verbena | anxiety |
| | | (8) | | Linalool (1.5) | | officinalis | |
| 17 | Oil | CBN | 1 | Myrcene (0.33) | | passiflora | insomnia |
| | | (4) | | caryophyllene | | | |
| | | | | (0.33) | | | |
| | | | | citronellol | | | |
| | | | | (0.33) | | | |
| 18 | Oil | THC | 4.1 | Linalool (1) | Terpineol (2) | mellisa | rage attack |
| | | and | | Terpineol (2) | | | |
| | | CBD | | Citronellol (1) | | | |
| | | (18) | | | | | |
| 19 | Oil | THC | 2 | Linalool (2) | Linalool (2) | Hypericum | self-injurious |
| | | (8) | | | | Perfortum | behavior |
| 20 | Oil | CBD | 2 | Limonene (2) | Limonene (2) | Witania | difficulty in |
| | | (10) | | | | | concentration |
| 21 | Oil | CBD | 4.2 | Limonene (3) | Limonene (3) | Sertraline | anxiety |
| | | (18) | | Linalool (1) | | | |
| 22 | Oil | THC | 3.2 | Terpineol (3) | Terpineol (3) | | Nighttime |
| | | (12) | | | | | composition |
| 23 | Oil | CBD | 2 | Pinene (2) | Pinene (2) | Clozapine | stress |
| | | (4) | | | | | |
| 24 | Tablet | THC | 2 | Limonene (2) | Limonene (2) | | stress |
| | | (8) | | | | | |
| 25 | Tablet | CBD | 1.5 | Humulene (1) | Humulene (1) | Rhodiola | difficulty in |
| | | (5) | | carene (0.5) | | | concentration |
| 26 | Tablet | THC | 3.2 | Linalool (2) | Linalool (2) | | Nighttime |
| | | (10) | | Bisabolol (1) | | | composition |
| 27 | Tablet | THC | 3 | Fenchol (1) | | Mentha piperita | Nausea |
| | | (10) | | Borneol (1) | | | |
| | | | | Myrcene (1) | | | |
| 28 | Tablet | CBD | 2.1 | Pinene (2) | Pinene (2) | Rivastigmine | attention |
| | | (10) | | | | | disorder |
| 29 | Tablet | THC | 4.1 | Myrcene (1) | Humulene (2) | Angelica arc | digestive |
| | | (12) | | germacrene (1) | | | problems |
| | | | | Humulene (2) | | | |
| 30 | Tablet | THC | 3.1 | Pinene (2) | Pinene (2) | whitania | motor tics |
| | | (10) | | Eucalyptol (0.5) | | | |
| | | | | Borneol (0.5) | | | |
| 31 | Tablet | CBD | 2 | Limonene (2) | Limonene (2) | Zingiber | seizures |
| | | (16) | | | | officinalis | |
| 32 | Tablet | CBD | 3.2 | Caryophyllene | Caryophyllene | Bacopa | difficulty in |
| | | (10) | | (3) | (3) | | concentration |
| 33 | Tablet | CBD | 2 | Linalool (2) | Linalool (2) | | hyperactivity |
| | | (5) | | | | | |
| 34 | Tablet | THC | 2 | Bisabolol (1.5) | Bisabolol (1.5) | Mellisa | insomnia |
| | | and | | Eucalyptol (0.5) | | | |
| | | CBN | | | | | |
| | | (8) | | | | | |
| 35 | Tablet | THC | 3.1 | Caryophyllene | Caryophyllene | Centella | vocal tics |
| | | and | | (2) | (2) | | |
| | | CBD | | Geraniol (0.5) | | | |
| | | (10) | | Menthol (0.5) | | | |
| | | | | Pinene (1) | | | |
| 36 | Cigarette | THC | 8.2 | Linalool (4) | Linalool (4) | | aggression |
| | | and | | Bisabolol (2) | | | |
| | | CBD | | Geraniol (2) | | | |
| | | (24) | | | | | |
| 37 | Cigarette | THC | 4.1 | Terpineol (3) | Terpineol (3) | Centella | self-injury |
| | | and | | Neral (1) | | | |
| | | CBD | | | | | |
| | | (14) | | | | | |
| 38 | Cigarette | THC | 6.2 | Terpineol (3) | Terpineol (3) | | insomnia |
| | | and | | Myrcene (2) | | | |
| | | CBD | | Citronellol (1) | | | |
| | | (22) | | | | | |

A-Form
B-Cannabinoid (content as % by weight)
C-Total Terpene (wt %)
D-Primary Terpene (content as % by weight)
E-Dedicated Terpene (wt %)
F-Additive
G-For treating
[1] Oil herein refers to a composition containing an extract of a cannabis plant material.

The scope of the invention shall include all modifications to, and variations of, the embodiments described herein that will be apparent to those skilled in the art, and other embodiments of the invention will be apparent to those skilled in the art from consideration of the specification and practice of the invention disclosed herein. It is intended that the embodiments described herein be considered only as exemplary embodiments of the invention.

What is claimed is:

1. A method of treatment of a condition associated with autism and/or an autism spectrum disorder selected from the group consisting of restlessness, rage attacks, agitation, treatment-resistant self-injurious behavior, stress, aggression, anxiety, irritability, behavioral outbreaks, communication problems, disruptive behavior, psychosis, hyperactivity, lethargy, seizure activity, hypersensitivity, stereotypy, inappropriate speech, sleep problems, insomnia, cognitive impairment, motor and/or vocal tics, digestive problems, lack of appetite, constipation, depression, incontinence, difficulty in concentration, attention disorder, poor learning capabilities, poor daily functioning, Asperger's syndrome, a pervasive developmental disorder in a not otherwise specified (PDD-NOS) subject, Rett syndrome, an intellectual disability, a condition characterized by language impairment, a condition characterized by social and communication disabilities, a condition characterized by unusual behaviors and interests, nervousness, nausea and vomiting, fatigue, deficits in balance, and combinations thereof in a subject in need thereof, the method comprising administering to the subject a therapeutically effective amount of a composition comprising
at least one cannabinoid; and
at least one primary terpene selected from the group consisting of limonene, eucalyptol, linalool, terpineol, caryophyllene, pinene, myrcene, humulene, citronellol, terpinen-4-ol, ocimene, geraniol, menthol, borneol, bisabolol, fenchol, guiaol and combinations thereof,
wherein said at least one primary terpene forms at least 20% of the total amount of terpenes in said composition,
wherein said at least one primary terpene optionally comprises a dedicated terpene which forms at least 40% by weight of the total amount of said primary terpene in the composition, and
wherein a total terpenes to cannabinoids weight/weight ratio in said composition is greater than 0.05:1.

2. The method of claim 1, wherein said composition comprises a dedicated terpene selected from the group consisting of humulene, caryophyllene, bisabolol, guaiol, pinene, linalool, terpineol, limonene, eucalyptol and combinations thereof, wherein said condition occurs during the daytime, wherein said condition is selected from the group consisting of including behavioral outbreaks, aggression, rage attacks, self-injurious behavior, irritability, restlessness, disruptive behavior, psychosis, hyperactivity, hypersensitivity, stereotypy, inappropriate speech, motor and/or vocal tics, communication problems, attention disorder, poor learning capabilities and poor daily functioning.

3. The method of claim 1, wherein said composition comprises a dedicated terpene selected from the group consisting of caryophyllene, bisabolol, pinene, linalool, terpineol and combinations thereof, wherein said condition comprises sleep problems.

4. The method of claim 1, wherein said condition is selected from the group consisting of Asperger's syndrome, a pervasive developmental disorder in a not otherwise specified (PDD-NOS) subject, Rett syndrome, an intellectual disability, a condition characterized by language impairment, a condition characterized by social and communication disabilities, a condition characterized by unusual behaviors and interests, and combinations thereof.

5. The method of claim 1, said composition further comprising at least one selected from the group consisting of bergamottin, apigenin, amentoflavone, quercetin and piperine and combinations thereof.

6. The method of claim 1, wherein said subject is a child.

7. The method of claim 1, wherein said subject is a female.

8. The method of claim 1, said composition further comprising a selected amount of a herbal preparation produced by at least one of distilling and extracting, wherein said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, lycium barbarum, Matricaria, Melissa officinalis, Mentha piperita, Mitaki, Mumio, Olea europea, origanum, Paeonia lactiflora, panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

9. The method of claim 1, wherein said at least one primary terpene is selected from the group consisting of limonene, eucalyptol, linalool, terpineol, myrcene, caryophyllene, citronellol, pinene, terpinene, nerolidol, eucalyptol, sabinene, pinene, limonene, borneol, fenchol, bisabolol, humulene, phytol, citral and combinations thereof,
wherein said composition further comprises an herbal preparation selected from the group consisting of extracts of Mellisa, Bacopa, Valeriane, Avena Sativa, passiflora, Humulus Lopus and combinations thereof, and
wherein said condition comprises insomnia and/or sleep problems.

10. The method of claim 9, wherein said composition further comprises a compound selected from the group consisting of Mirtazapine, melatonin, Donepezil and combinations thereof.

11. The method of claim 1, wherein said at least one cannabinoid comprises CBD and optionally THC at a CBD to THC weight/weight ratio greater than 1:1, wherein said primary terpene is selected from the group consisting of limonene, eucalyptol, linalool, terpineol, pinene, pulegone, cymene, ocimene, caryophyllene, humulene, carene and combinations thereof,
wherein said compositions further comprises an herbal preparation selected from the group consisting of extracts of Bacopa, Centella, Witania, Rhodiola and combinations thereof, and
wherein said condition is selected from the group consisting of difficulty in concentration, cognitive impairment and attention disorder and combinations thereof.

12. The method of claim 11, wherein said composition further comprises a compound selected from the group consisting of Methylphenidate, Venlafaxine Memantine, rivastigmine and combination thereof.

13. The method of claim 1, wherein said at least one cannabinoid comprises CBD and optionally THC at a CBD to THC weight/weight ratio greater than 1:1, wherein said primary terpene is selected from the group consisting of limonene, eucalyptol, linalool, terpineol, citronellol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene and combinations thereof,
wherein said composition further comprises an herbal preparation selected from the group consisting of extracts of Hypericum Perfortum, Avena Sativa, Passiflora, Humulus Lopus and combinations thereof, and said administering is performed during nightime; or
wherein said composition comprises an herbal preparation selected from the group consisting of extracts of Hypericum Perfortum, Centella, Bacopa, mellisa, Verbena officinalis and combinations thereof, and wherein said administering is performed during daytime, and
wherein said condition is selected from the group consisting of anxiety, irritability, nervousness, restlessness, stress, aggression, hyperactivity, disruptive behavior, psychosis, behavioral outbreaks, hypersensitivity, rage attack, agitation, treatment-resistant self-injurious behavior, communication problems, depression and combinations thereof.

14. The method of claim 1, wherein said at least one cannabinoid comprises THC and optionally CBD at a THC to CBD weight/weight ratio greater than 1:1, wherein said at least one primary terpene is selected from the group consisting of limonene, eucalyptol, linalool, terpineol, citronellol, geraniol, menthol, humulene, bisabolol, borneol, fenchol, pinene, caryophyllene, myrcene, cymene, terpinene and combinations thereof,
wherein said composition further comprisies an herbal preparation selected from the group consisting of extracts of Atractylodes macrocephala, Angelica arc, Mentha piperita, Matricaria, Zingiber officinalis and combinations thereof, and
wherein said condition is selected from the group consisting of lack of appetite, nausea and vomiting and combinations thereof.

15. The method of claim 1, wherein said at least one primary terpene is selected from the group consisting of limonene, eucalyptol, linalool, terpineol, cymene, ocimene, terpinene, caryophyllene, citronellol, sabinene, pinene, myrcene, germacrene, geraniol, nerolidol, menthol, humulene, fenchol, borneol and combinations thereof,
wherein said composition further comprises an herbal preparation selected from the group consisting of extracts of Aloe spp, Angelica arc, Rumex, Silybum marianum, Rehmania glutinosa, Taraxacum, Humulus lupus, Verbena officinalis and combinations thereof, and
wherein said condition is selected from the group consisting of digestive problems, incontinence and constipation and combinations thereof.

16. The method of claim 1, wherein said primary terpene is selected from the group consisting of limonene, eucalyptol, linalool, terpineol, myrcene, pinene, nerolidol, caryophyllene, geraniol, menthol, borneol, bisabolol, fenchol and combinations thereof;
wherein said composition further comprises an herbal preparation selected from the group consisting of extracts of Cypripedium pubescens, whitania, Hypericum, Rhodiola, Vallerian, passiflora, Bacopa, Centella, Rhodiola and combinations thereof,
wherein said administering is performed during daytime, and
wherein said condition is selected from the group consisting of stereotypy, inappropriate speech, motor tics and/or vocal tics and combinations thereof.

17. The method of claim 1, wherein said at least one cannabinoid comprises CBD and optionally THC at a CBD to THC weight/weight ratio greater than 1:1, wherein said primary terpene is selected from the group consisting of limonene, eucalyptol, linalool, terpineol ocimene, myrcene, limonene, caryophyllene and combinations thereof,
wherein said composition further comprises an herbal preparation selected from the group consisting of extracts of Bacopa, centella, Curcuma, Whitania, Zingiber officinalis, Peonia and combinations thereof, and
wherein said condition comprise seizure activity.

18. The method of claim 1, wherein said at least one cannabinoid comprises CBD and optionally THC at a CBD to THC weight/weight ratio of greater than 1:1, wherein said primary terpene is selected from the group consisting of limonene, eucalyptol, linalool, terpineol pinene, terpinene, myrcene, caryophyllene, borneol and combinations thereof,
wherein said composition further comprises an herbal preparation selected from the group consisting of extracts of centella, Rhodiola, Whitania, Astragalus membranous, Bacopa, Ganoderma and combinations thereof.
wherein said condition is selected from the group consisting of lethargy, fatigue and deficits in balance and combinations thereof.

19. The method of claim 1, wherein said composition comprises a compound selected from the group consisting of Mirtazapine, melatonin, Donepezil, Methylphenidate, Venlafaxine Memantine, rivastigmine, Risperidone, Aripiprazole, Clozapine, Haloperidol, Sertraline, Oxytocin, Secretin, Fluoxetine, Citalopram, Bumetanide, Acamprosate, Atomoxetine, baclofen, DMXB-A (a derivative of the nemertine toxin anabasein), EPI-743 (vincerinone), RG7314 (Roche), Divalproex, Dextromethorphan hydrobromide, and/or quinidine sulfate and combinations thereof.
